(19) United States
(12) Patent Application Publication
(10) Pub. No.: US 2022/0387349 A1
(43) Pub. Date: Dec. 8, 2022

(54) TREATMENT OF CUTANEOUS ADVERSE EFFECTS CAUSED BY ONCOLOGICAL THERAPY WITH TOPICAL TAPINAROF COMPOSITIONS

(71) Applicant: Sol-Gel Technologies Ltd, Ness Ziona (IL)

(72) Inventors: Moshe ARKIN, Kfar Shmaryahu (IL); Marcel ZIGHELBOIM, Kiryat Motzkin (IL)

(73) Assignee: Sol-Gel Technologies Ltd, Ness Ziona (IL)

(21) Appl. No.: 17/763,231

(22) PCT Filed: Sep. 24, 2020
(86) PCT No.: PCT/IL2020/051053

§ 371 Date: Mar. 24, 2022

Related U.S. Application Data
(60) Provisional application No. 62/906,303, filed on Sep. 26, 2019.

(51) Int. Cl. A61K 31/05; A61K 9/00; A61K 47/20; A61K 47/10; A61K 47/22; A61K 47/14; A61K 9/10; A61K 47/02; A61K 47/32; A61K 47/12; A61K 47/18; A61K 9/107; A61K 47/44; A61K 47/06; A61K 9/06; A61K 47/26; A61K 47/24

(52) U.S. Cl. CPC A61K 31/05; A61K 9/0014; A61K 47/20; A61K 47/10; A61K 47/22; A61K 47/14; A61K 9/10; A61K 47/02; A61K 47/32; A61K 47/12; A61K 47/186; A61K 9/107; A61K 47/44; A61K 47/06; A61K 9/06; A61K 47/26; A61K 47/183; A61K 47/24

(57) ABSTRACT

Provided herein is a topical composition comprising tapinarof and methods of treatment of cutaneous adverse effects caused by oncological therapy with the tapinarof composition. The tapinarof compositions of this invention are useful for the treatment, prevention or amelioration of cutaneous and/or mucosal adverse effect caused by oncological therapy selected from chemotherapy, immunotherapy, radiation therapy, targeted therapy, hormone therapy and stem cell transplant therapy.

### FIELD OF THE INVENTION

The present invention, in some embodiments thereof, relates to methods of treatment of cutaneous adverse effects caused by oncological therapy with topical tapinarof compositions. The tapinarof compositions of this invention are useful for the treatment, prevention or amelioration of cutaneous and/or mucosal adverse effect caused by oncological therapy selected from chemotherapy, immunotherapy, radiation therapy, targeted therapy, hormone therapy and stem cell transplant therapy.

### BACKGROUND

Oncological therapy, such as chemotherapy, immunotherapy, radiation therapy, targeted therapy, hormone therapy and stem cell transplant therapy, is a staple of cancer treatment, alongside surgical oncology.

More than 500 distinct kinds of adverse effects (AEs) have been associated with modern cancer therapy and a grading system (CTCAE) has been developed for the assessment of their severity (Trotti A. et al., Seminars in Radiation Oncology, Vol. 13, No. 3, July 2009, pp. 176-181). One of the types of the oncological AEs are the cutaneous AEs caused by oncological treatment.

### SUMMARY OF THE INVENTION

This invention provides a topical composition comprising tapinarof and a carrier suitable for topical administration. The compositions may further comprise an emollient and/or a penetration enhancer.

Also provided are methods for the treatment, prevention or amelioration of a cutaneous and/or mucosal adverse effect caused by oncological therapy, selected from chemotherapy, immunotherapy, radiation therapy, targeted therapy, hormone therapy and stem cell transplant therapy by topical administration of a composition of this invention to an oncological therapy patient in need thereof.

### DETAILED DESCRIPTION OF THE INVENTION

The present invention provides novel topical tapinarof compositions and methods of treatment, prevention and amelioration of a cutaneous and/or mucosal adverse effect caused by oncological therapy, selected from chemotherapy, immunotherapy, radiation therapy, targeted therapy, hormone therapy and stem cell transplant therapy by topical administration of a composition of this invention to a patient in need thereof, which underwent oncological treatment.

The topical combination composition of this invention comprises from about 0.01% w/w to about 10.0% w/w tapinarof and a carrier suitable for topical administration.

Tapinarof is presently indicated for topical use at 1% w/w, according to clinical studies (vide infra). Higher than 1% w/w tapinarof concentrations exhibit undesirable side-effects.

The undesirable tapinarof side-effects at concentrations equal to or higher than 1% w/w are avoided or minimized in the present invention by a special encapsulation process, detailed in Examples 1 and 2.

It occurred to the present inventors, that topical tapinarof compositions comprising tapinarof may enable treatment, prevention and amelioration of a cutaneous and/or mucosal adverse effect caused by oncological therapy, selected from chemotherapy, immunotherapy, radiation therapy, targeted therapy, hormone therapy and stem cell transplant therapy.

Without wishing to be bound by theory, the tapinarof beneficial effect for the treatment, prevention and amelioration of a cutaneous and/or mucosal adverse effect caused by oncological therapy, is due to the anti-inflammatory and anti-oxidant properties of tapinarof.

The tapinarof topical composition comprises from about 0.01% w/w to about 10.0% w/w tapinarof and a carrier and optionally with an anti-oxidant suitable for topical administration.

Tapinarof in the above composition is encapsulated using the process detailed in Example 1 or 2, for preventing side-effects at concentrations equal to or higher than 1% w/w.

Optionally, the above tapinarof composition may further comprise a moisturizer, urea, ammonium lactate, an anti-oxidant or combinations thereof and/or a penetration enhancer

The penetration enhancer is selected from dimethylsulfoxide (DMSO), methylsulfonylmethane (MSM), propylene glycol, dimethyl isosorbide, isopropyl myristate and combinations thereof.

There is an unmet need for methods of treatment, prevention and amelioration of a cutaneous and/or mucosal adverse effect caused by oncological therapy, devoid of side-effects.

The active agent in the composition of this invention is detailed below.

### Tapinarof

Tapinarof (3,5-dihydroxy-4-isopropyl-trans-stilbene, benvitimod, GSK2894512) is a first-in-class drug, whose mechanism is not yet fully understood. It is being developed by Glaxo Smith Kline (Stiefel, a GSK company) and Dermavant as a topical drug for treatment of mild to moderate psoriasis and atopic dermatitis. It was shown in both mouse models and in vitro human skin studies to inhibit specific proinflammatory mediators, including interleukin-6 and interleukin-17A, and enhance skin barrier function (J Invest Dermatol. 2017 October;137[10]:2110-9).

According to Jancin B. (Dermatology News, Nov. 11, 2017), in the above studies, the 1% tapinarof arm showed higher efficacy and had a quicker onset of action than the 0.5% arm or vehicle. The most frequent adverse events associated with tapinarof were folliculitis and contact dermatitis.

Tapinarof, which seems to be a significant advance in psoriasis treatment, presents however higher adverse effects (44.5%) when compared to placebo (20.2%) and calcipotriene (19.5%) as a single drug (Medscape Mar. 5, 2017 report by Frellick M., on Abstr. 5629, Amer. Acad. of Dermatology meeting, Mar. 4, 2017).

There is an unmet need for methods for the treatment of cutaneous adverse effects caused by oncological therapy, using tapinarof topical compositions, devoid of serious side-effects.

### Tapinarof Compositions

Provided herein are compositions, combinations, kits and articles of manufacture comprising tapinarof, for treating a cutaneous and/or mucosal adverse effect caused by oncological therapy. The compositions, combinations and articles of manufacture can be administered using a variety of routes such as topical application or transdermal application. The preferred route is the topical route and the preferred formulations are the cream, the gel and the lotion.

Therapeutically effective amounts of tapinarof are mixed with a suitable pharmaceutical carrier or vehicle suitable and optionally an anti-oxidant for topical use, for the treatment, prevention or alleviation of the cutaneous and/or mucosal adverse effect caused by oncological therapy.

Tapinarof is included in the composition of this invention in an amount effective for treating, preventing or alleviating the cutaneous and/or mucosal adverse effect caused by oncological therapy. The concentration of the active agents in the composition will depend on absorption, inactivation, excretion rates of the active compound, the dosage schedule, and amount administered as well as other factors known to those of skill in the art. The dosage and regimen of administration may be determined by dose finding studies, as known in the art.

Exemplary dosages, strengths and concentrations of tapinarof in the topical compositions, are in the range of from about or at 0.01%, 0.1%, 0.25%, 0.5%, 1%, 2%, 3%, 4%, 5%, 6%, 7%, 8%, 9% or 10% w/w. Typical strengths in the topical combination compositions of this invention are 0.25%, 0.5% or 1% w/w tapinarof.

The frequency of administration of the composition of this invention can be determined empirically. Exemplary frequencies are once daily, twice daily, weekly, bi-weekly or monthly. Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the cutaneous adverse effects caused by oncological therapy. For example, dosage administration can begin at from twice a day, to once a day, to two times a week, to once a week, to once every two weeks or less frequent than once every two weeks.

Pharmaceutical carriers or vehicles suitable for preparation of the compositions provided herein include any such carriers known to those skilled in the art to be suitable for the particular mode of administration.

In some embodiment, the compositions of this invention comprises a carrier, a solvent, an emollient, a surfactant, an anti-oxidant, a chelating agent, a gelling agent, a wetting agent, a penetration enhancer, a preservative, an anti-oxidant, a buffer or any combination thereof.

In some embodiments, the compositions of this invention comprise a penetration enhancer. In another embodiment, the penetration enhancer is selected from the group consisting of dimethylsulfoxide (DMSO), diethylene glycol monoethyl ether (Transcutol), methylsulfonylmethane (MSM), oleic acid, oleyl alcohol, propylene glycol, dimethyl isosorbide, isopropyl myristate, diethyl sebacate, ethanol, isopropyl alcohol, a polyethylene glycol, hexylene glycol, glycofurol and any combination thereof.

In some embodiments, the compositions of this invention comprise an emollient. In another embodiment, the emollient is selected from the group consisting of castor oil, mineral oil, vegetable oil, soybean oil, shea butter, cocoa butter, paraffin, beeswax, oleic acid, squalene, cetyl alcohol, isopropyl myristate, urea, glycerol, propylene glycol, lactic acid and any combination thereof.

In some embodiments, the compositions of this invention comprise a chelating agent. In another embodiment, the chelating agent is selected from the group consisting of EDTA deferoxamine, deferiprone, deferasirox, dimercaptosuccinic acid (succimer) triethylenetetramine (trientine) and any combination thereof.

In some embodiments, the compositions of this invention comprise an anti-oxidant. In another embodiment, the anti-oxidant is selected from the group consisting of butylated hydroxytoluene (BHT), parabens, propyl gallate, ascorbic acid, flavones, flavanones, flavonols, stilbenoids, gallic acid, cinnamic acid, ellagic acid, salicylic acid, curcumin, eugenol, citric acid and any combination thereof.

In some embodiments, the compositions of this invention comprise a solvent. In another embodiment the solvent is selected from the group consisting of propylene glycol, diethylene glycol monoethyl ether (Transcutol), dimethylsulfoxide (DMSO), diethyl sebacate, polyethylene glycol, oleyl alcohol, isosorbide dimethyl ether, ethanol, isopropyl alcohol, isopropyl myristate, oleic acid, hexylene glycol, glycerin, glycofurol and any combination thereof.

In some embodiments, the compositions of this invention comprise a preservative. In another embodiment the preservative is selected from the group consisting of benzoic acid, methylparaben, chlorocresol, Phenoxyethanol, propyl paraben, benzalkonium chloride, benzyl alcohol, imidazolidinyl urea, sodium benzoate, sorbic acid, thimerosal sorbic acid, sodium sorbate, sodium sulfite, ethanol, tocopherols and any combination thereof.

In some embodiments, the compositions of this invention comprise a buffer. In another embodiment, the buffer is selected from the group consisting of citric acid, phosphoric acid, acetic acid, tartaric acid, glutamic acid, aspartic acid, malic acid, succinic acid, fumaric acid, salt thereof, and any combination thereof.

In some embodiments, the compositions of this invention comprise a gelling agent. In another embodiment, the gelling agent is selected from the group consisting of carbomer homopolymer type A (Carbopol®981, Carbopol®980), Sepineo P600, Natrosol hydroxyethylcellulose, starch, pectin, gelatin, agar, alginic acid and salts thereof, Carbomer® 934, carboxymethylcellulose, hydroxypropylmethylcellulose and any combination thereof.

In some embodiments, the compositions of this invention comprise a surfactant. In another embodiment, the surfactant is selected from the group consisting of carbomer copolymer type B (Pemulen®TR-1), sorbitan monooleate (Span80), polysorbate 80 (Tween 80), polysorbate 20, polysorbate 60 (Tween 60), sodium dodecylsulfate, cetearyl alcohol, dioctyl sodium sulfosuccinate, glyceryl oleate, glyceryl stearate, poloxamers and any combination thereof.

In some embodiments, the compositions of this invention comprise a wetting agent. In another embodiment, the wetting agent is selected from the group consisting of PEG 400, glycerin, poloxamers, sorbitan monooleate (Span80), polysorbate 80 (Tween 80), polysorbate 20, polysorbate 60 (Tween 60), benzalkonium chloride, sodium dodecylsulfate and any combination thereof.

The resulting composition may be a cream, a lotion, a gel, an ointment, an emulsion, a solution, a suspension, an elixir, a tincture, a paste, a foam, an aerosol, a spray, a patch, a transdermal patch, a powder, a shake lotion, a solid, a sponge, a tape, a shampoo and an applicator syringe (for intradermal injection), or any other formulation suitable for topical administration. The preferred compositions are the cream, the gel and the lotion.

Pharmaceutical carriers or vehicles suitable for administration of the compounds provided herein include any such carriers known to those skilled in the art to be suitable for the particular mode of administration. In addition, the compounds may be formulated as the sole pharmaceutically active ingredient in the composition or may be combined with other active ingredients. The active agents are included in the carrier in an amount sufficient to exert a therapeutically useful effect i.e., amelioration of the symptoms of the oncological therapy cutaneous adverse effects, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include a cream, a lotion, a gel, an ointment, an emulsion, a solution, a suspension, an elixir, a tincture, a paste, a foam, an aerosol, a spray, a patch, a transdermal patch, a powder, a shake lotion, a solid, a sponge, a tape, a shampoo or an applicator syringe (for intradermal injection). Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

### Methods of Treatment

According to an aspect of the invention, there is provided a method of treatment of a cutaneous and/or mucosal adverse effect caused by oncological therapy by administration to an oncological therapy subject in need thereof a tapinarof composition.

In some embodiments, the effective amount is a therapeutically effective amount of tapinarof, namely an amount which will cure, treat, alleviate or prevent a cutaneous and/or mucosal adverse effect caused by oncological therapy.

### Regimen of Administration of the Tapinarof Topical Compositions

Therapeutically effective concentrations of tapinarof in the compositions of this invention for treatment, prevention or alleviation of the symptoms of a cutaneous and/or mucosal adverse effect caused by oncological therapy are determined by empirical methods known in the art.

The dosage and regimen of administration may be determined by dose finding studies, as known in the art.

Exemplary dosages, strengths and concentrations of tapinarof in the topical compositions of this invention, are in the range of from about or at 0.01%, 0.05%, 0.1%, 0.25%, 0.5%, 1%, 2%, 3%, 4%, 5%, 6%, 7%, 8%, 9% or 10% w/w. Typical strengths of tapinarof in the topical compositions of this invention are 0.25%, 0.5% or 1% w/w tapinarof.

The frequency of administration can be determined empirically. Exemplary frequencies are once daily, twice daily, weekly, bi-weekly or monthly.

Typical administration frequencies of the topical compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the cutaneous adverse effects caused by oncological therapy. For example, dosage administration can begin at from twice a day, to once a day, to two times a week, to once a week, to once every two weeks or less frequent than once every two weeks.

### Kits

Kits are provided, containing the composition of this invention, optionally including instructions for administration.

The compositions provided herein can be packaged as articles of manufacture containing packaging material, a composition provided herein, and a label that indicates that the composition is for treating a cutaneous adverse effect and is formulated for topical delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes and any packaging material suitable for a selected formulation and intended mode of administration and treatment.

### Embodiments

In some embodiments, there is provided a topical composition for the treatment, prevention or alleviation of a cutaneous and/or mucosal adverse effect caused by oncological therapy selected from chemotherapy, immunotherapy, radiation therapy, targeted therapy, hormone therapy and stem cell transplant therapy comprising from about 0.01% to about 1% w/w, from about 1% to about 3% w/w, from about 1% to about 5% w/w tapinarof, from about 3% to about 5% w/w tapinarof, or from about 5% to about 10% w/w tapinarof and a carrier suitable for topical administration.

In some embodiments, there is provided a topical composition for the treatment, prevention or alleviation of a cutaneous and/or mucosal adverse effect caused by oncological therapy comprising tapinarof, wherein tapinarof is encapsulated using the process detailed in Example 1 or 2, or non-encapsulated as in Examples 3 or 4, for preventing side-effects at concentrations equal to or higher than 1% w/w.

In some embodiments, there is provided a topical composition for the treatment, prevention or alleviation of a cutaneous and/or mucosal adverse effect caused by oncological therapy comprising tapinarof, further comprising an emollient, a moisturizer, urea, ammonium lactate, an anti-oxidant or combinations thereof and/or a penetration enhancer.

In some embodiments, there is provided a topical composition for the treatment, prevention or alleviation of a cutaneous and/or mucosal adverse effect caused by oncological therapy comprising tapinarof, further comprising a moisturizer, urea, ammonium lactate or combinations thereof and/or a penetration enhancer, wherein the penetration enhancer is selected from DMSO, MSM, propylene glycol, dimethyl isosorbide, isopropyl myristate and combinations thereof.

In some embodiments, there is provided a dosage form comprising the tapinarof composition of this invention, wherein the composition is formulated in a dosage form selected from Suitable pharmaceutically and dermatologically acceptable vehicles for topical application include a cream, a lotion, a gel, an ointment, an emulsion, a solution, a suspension, an elixir, a tincture, a paste, a foam, an aerosol, a spray, a patch, a transdermal patch, a powder, a shake lotion, a solid, a sponge, a tape, a shampoo and an applicator syringe (for intradermal injection).

In some embodiments, there is provided a method of treatment, prevention or alleviation of a cutaneous adverse effect caused by oncological therapy selected from chemotherapy, immunotherapy, radiation therapy, targeted therapy, hormone therapy and stem cell transplant therapy, by topical administration to an oncological therapy subject in need thereof of a therapeutically effective amount of a topical composition comprising from about 0.01% to about 1% w/w, from about 1% to about 3% w/w, from about 1% to about 5% w/w, from about 3% to about 5% w/w tapinarof, or from about 5% to about 10% w/w tapinarof and a carrier suitable for topical administration and optionally an emollient, a moisturizer, urea, ammonium lactate or combinations thereof and/or a penetration enhancer.

In some embodiments, there is provided a method of treatment, prevention or alleviation of a cutaneous adverse effect caused by oncological therapy selected from chemotherapy, immunotherapy, radiation therapy, targeted therapy, hormone therapy and stem cell transplant therapy, by topical administration to an oncological therapy subject in need thereof of a therapeutically effective amount of a topical composition comprising from about 0.01% to about 1% w/w, from about 1% to about 3% w/w, from about 1% to about 5% w/w, from about 3% to about 5% w/w tapinarof, or from about 5% to about 10% w/w tapinarof and a carrier suitable for topical administration and optionally an emollient, a moisturizer, urea, ammonium lactate or combinations thereof and/or a penetration enhancer, wherein the cutaneous adverse effect is selected from maculo-papular rashes, inflammatory rashes, photosensitivity and secondary skin tumors.

In some embodiments, there is provided a method of treatment, prevention or alleviation of a cutaneous adverse effect caused by oncological therapy selected from chemotherapy, immunotherapy, radiation therapy, targeted therapy, hormone therapy and stem cell transplant therapy, by topical administration to an oncological therapy subject in need thereof of a therapeutically effective amount of a topical composition comprising from about 0.01% to about 1% w/w, from about 1% to about 3% w/w, from about 1% to about 5% w/w, from about 3% to about 5% w/w tapinarof, or from about 5% to about 10% w/w tapinarof and a carrier suitable for topical administration and optionally an emollient, a moisturizer, urea, ammonium lactate or combinations thereof and/or a penetration enhancer, wherein the cutaneous adverse effect is caused by cutaneous inflammation.

In some embodiments, there is provided a method of treatment, prevention or alleviation of a cutaneous adverse effect caused by oncological therapy selected from chemotherapy, immunotherapy, radiation therapy, targeted therapy, hormone therapy and stem cell transplant therapy, by topical administration to a subject in need thereof of a therapeutically effective amount of a topical composition comprising from about 0.01% to about 1% w/w, from about 1% to about 3% w/w, from about 1% to about 5% w/w, from about 3% to about 5% w/w tapinarof, or from about 5% to about 10% w/w tapinarof and a carrier suitable for topical administration and optionally an emollient, a moisturizer, urea, ammonium lactate or combinations thereof and/or a penetration enhancer, wherein the cutaneous adverse effect is caused by the upregulation of pro-inflammatory cytokines and/or chemokines.

In some embodiments, there is provided a method of treatment, prevention or alleviation of a cutaneous adverse effect caused by oncological therapy selected from chemotherapy, immunotherapy, radiation therapy, targeted therapy, hormone therapy and stem cell transplant therapy, by topical administration to an oncological therapy subject in need thereof of a therapeutically effective amount of a topical composition comprising from about 0.01% to about 1% w/w, from about 1% to about 3% w/w, from about 1% to about 5% w/w, from about 3% to about 5% w/w tapinarof, or from about 5% to about 10% w/w tapinarof and a carrier suitable for topical administration and optionally an emollient, a moisturizer, urea, ammonium lactate or combinations thereof and/or a penetration enhancer, wherein the administration comprises once or twice daily topical application of therapeutically effective amounts of the said composition to the skin portion of the oncological therapy subject affected by the said cutaneous adverse effect until the cutaneous adverse effect is cured, prevented or alleviated or according to doctor's instructions.

In some embodiments, there is provided a regimen of administration comprising the once daily or twice daily administration to an oncological therapy subject in need thereof of a therapeutically effective dose of the composition comprising from about 0.01% to about 1% w/w, from about 1% to about 3% w/w, from about 1% to about 5% w/w, from about 3% to about 5% w/w tapinarof, or from about 5% to about 10% w/w tapinarof and a carrier suitable for topical administration and optionally an emollient, a moisturizer, urea, ammonium lactate or combinations thereof and/or a penetration enhancer, until the cutaneous adverse effect is cured, prevented or alleviated or according to doctor's instructions.

In some embodiments, there is provided a regimen of administration comprising the once daily or twice daily administration to an oncological therapy subject in need thereof a therapeutically effective amount of the dosage form of this invention until the cutaneous adverse effect is cured, prevented or alleviated or according to doctor's instructions.

In some embodiments, there is provided a kit comprising one or more dosage forms of this invention and instructions for use.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or” treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to an oncological therapy subject in need thereof.

As used herein, the term “essentially free” generally refers to a composition having less than about 2 percent by weight, more preferably 1 percent per weight, less than about 0.5 percent by weight or even less than 0.1 percent by weight of a certain ingredient, based on the total weight of the composition.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader.

All the information contained therein is incorporated herein by reference.

In the examples below, all % values referring to a solution are in (w/w).

All % values, referring to dispersions (suspensions) are in (w/w).

Unless otherwise indicated, all solutions used in the example below refer to an aqueous solution of the indicated ingredient.

All the exemplary compositions comprise micronized tapinarof.

Tapinarof in the exemplary compositions may be used as such as detailed in Examples 3 or 4, or encapsulated as detailed in Examples 1-2.

### Example 1: Preparation of Silicone Dioxide (SiO₂) Encapsulated Tapinarof Dispersed in Water (15% w/w Tapinarof)

### Preparation of Tapinarof Dispersion and Acid Cocktail

Tapinarof dispersion is prepared by mixing 378 grams of CTAC CT-429 (Cetrimonium Chloride 30%), 6,756 grams of micronized tapinarof having an average particle size of less than 1 μm, and 18,855 grams water under high shear. The dispersion is homogenized for 60 min at 33° C. (no more than 45° C.).

An acid cocktail is prepared using 1013 grams Hydrochloric Acid (37%), 215.3 grams anhydrous Citric Acid, 322.3 grams Lactic Acid (90%), and 1632 grams water.

Coating Cycle

The coating cycle is started by adding 953 grams sodium silicate solution extra pure (28%) to the tapinarof dispersion prepared in step a) under high shear, followed by adding the acid cocktail prepared in step (a) and followed by adding 1675 grams PDAC (3%) solution to the mixture. The cycle is repeated another 5 times. After the 6 cycles, the pH of the mixture is adjusted to 5.0 using the acid cocktail, and water is added to complete the total weight of the mixture to 45 kilograms.

The composition of the final tapinarof water suspension product is shown in Table 1.

TABLE 1
Composition of the encapsulated
tapinarof 15% water suspension

| | % w/w of ingredient |
|---|---|
| Ingredient | in the suspension |
| Polyquarternium-7 | 5.6 |
| Hydrochloric Acid 37% | 2.0 |
| Citric Acid, Anhydrous | 0.4 |
| Lactic Acid | 0.6 |
| Silicone Dioxide | 3.4 |
| Sodium Hydroxide | 1.4 |
| Cetrimonium Chloride | 0.84 |
| Tapinarof | 15.00 |
| Sterile Water for Irrigation | Up to 100% |

The above composition may be diluted to the required tapinarof concentration (e.g. 1% w/w/or 5% w/w) by addition of carrier.

### Example 2

### Preparation of Encapsulated Tapinarof Dispersed in Oil (3.06% w/w Tapinarof)

a) Oil Phase

45.9 grams of micronized tapinarof having an average particle size of less than 1 μm are mixed in 129.3 grams of Squalane. 86.16 grams of Tetraethoxysilane (TEOS) are added, and the resulted mixture was milled at 5000 rpm in a ball mill for 10 minutes with an upper propeller mixer at a speed of 250 rpm for 7 minutes, followed by 400 rpm for 3 minutes. 140.4 grams of milled tapinarof in oil is aliquoted out and then heated to 60° C. 9.0 grams of Beeswax are added and melted in the oil phase.

b) Water Phase

3.3 grams CTAC (Cetrimonium Chloride) are dissolved in 490.0 g water at 60° C. Unless indicated otherwise, in all examples described herein, the term “water” refers to sterile water for irrigation (USP).

c) Core-Shell Step

124.5 grams of the oil phase prepared in step (a) is added to the water phase and homogenized at 4000 rpm for 1 minute. 17.9 grams of sodium silicate extra pure solution (28%) are added to the emulsion. The pH of the emulsion is adjusted to 4.0 using HC15N solution. Water is added to complete the total weight of the mixture to 650 grams. The suspension is then stirred for 17 hours at 25° C. for the TEOS hydrolysis to be completed. The composition of the final encapsulated tapinarof water suspension product is shown in Table 2.

TABLE 2
Composition of the encapsulated
tapinarof 3.06% water suspension

| | % of pure ingredient |
|---|---|
| Ingredient | in the suspension |
| Beeswax | 1.15 |
| Squalane | 8.62 |
| Tetraethoxysilane | 5.74 |
| Tapinarof | 3.06 |
| Cetrimonium Chloride | 0.15 |
| Sodium hydroxide | 0.74 |
| Hydrochloric acid | 0.40 |
| Sterile Water for Irrigation | Up to 100% |

The above composition may be diluted to the required tapinarof concentration (e.g. 1% w/w/or 2% w/w) by addition of carrier.

### Example 3: Preparation of a Non-Encapsulated Tapinarof Composition

The topical tapinarof cream consists of:

0.25-3.0% w/w tapinarof,

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

Adjust to pH 6.0-7.0 with.1M NaOH or 0.1M HCl.

The cream composition is prepared by the following steps:

(1) weigh tapinarof having an average particle size of less than lum;

(2) heat the propylene glycol to 60° C. in a water bath;

(3) add to the heated propylene glycol while stirring tapinarof, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof cream in an aluminum tube or other delivery system.

### Example 4: Preparation of Tapinarof, 1% Lotion

| Raw Material | | |
|---|---|---|
| (compendial Name) | % w/w | Function |
| Tapinarof 98% | 1.0 | Active |
| Castor oil | 4.0 | Emollient |
| Mineral oil light | 4.0 | Emollient |
| Diethylene glycol | 5.5 | Solvent, |
| monoethyl | | penetration agent |
| ether (Transcutol) | | |
| Dimethyl Sulfoxide | 5.5 | Solvent, |
| | | penetration agent |
| Sorbitan Monooleate | 0.1 | Surfactant |
| (Span 80) | | |
| Propylene glycol | 10.0 | Solvent |
| Disodium Edetate | 0.1 | Chelating agent |
| (EDTA) | | |
| Carbomer | 0.4 | Surfactant |
| Copolymer Type | | |
| B Pemulen ®TR-1 | | |
| Carbomer | 0.6 | Gelling |
| Homopolymer | | |
| Type A Carbopol ®981 | | |
| Purified water | 64.00 | Continuous phase |
| Benzoic Acid | 0.25 | Preservative |
| BHT | 0.1 | Anti-oxidant |
| Citric Acid | 0.1 | Buffer |
| Sodium Citrate | 0.2 | Buffer |
| Sodium hydroxide | For pH | pH adjustment |
| pellets | adjustment | |
| Purified water | q.s. to 100 | Continuous phase |

### Water Phase

Into a glass beaker water and Benzoic Acid were added. The beaker was placed inside a hot water bath adjusted to 60° C. and the mixture was mixed with a magnetic stirrer until a clear solution free from particles was obtained. Then EDTA, Citric Acid and Sodium Citrate were added. The mixing was continued until a clear solution was obtained. The solution was cooled down to room temperature. Then, the pH was slowly adjusted to pH 6.0 with NaOH 20%.

### Oil Phase

In a separate glass beaker Mineral oil light, castor oil, span 80 and BHT were weighed. The beaker was placed inside a hot water bath adjusted to 60° C. and the mixture was mixed with a magnetic stirrer until a uniform solution was obtained. Then Carbopol®981 and Pemulen®TR-1 were slowly added and the mixing was continued until a homogenous mixture was obtained. The mixture was cooled down to room temperature.

### Active Phase

Into a separate glass beaker Propylene Glycol, Transcutol and DMSO were weighed. The mixture was mixed with a magnetic stirrer until a uniform homogenous solution was obtained. The beaker was covered with an aluminum foil and placed in a yellow light hood. Tapinarof was slowly added, and the mixing was continued for about 1 h until a clear solution free from particles was obtained.

The oil phase was slowly added to the water phase while homogenizing for about 5 minutes, until there were no lumps. Then, the active phase was slowly added to the Water+Oil phase while homogenizing for about 5 minutes.

Water was added for batch completion, and final pH was measured to conform it is around pH 5.

Each of the above compositions of Examples 1-4 may further comprise an emollient, a moisturizer, urea, ammonium lactate or combinations thereof and/or a penetration enhancer selected from DMSO, MSM, propylene glycol, dimethyl isosorbide, isopropyl myristate and combinations thereof.

1. A method of treatment, prevention or alleviation of a cutaneous and/or mucosal adverse effect caused by oncological therapy selected from chemotherapy, immunotherapy, radiation therapy, targeted therapy, hormone therapy and stem cell transplant therapy, wherein the method comprises administering to an oncological therapy subject in need thereof a topical composition comprising from about 0.01% to about 1% w/w, from about 1% to about 3% w/w, from about 1% to about 5% w/w, from about 3% to about 5% w/w tapinarof, or from about 5% to about 10% w/w tapinarof and a carrier suitable for topical administration.

2. The method of claim 1, wherein tapinarof is encapsulated, for preventing side-effects at concentrations higher than 1% w/w.

3. The method of claim 1, wherein the topical composition further comprising an emollient, a moisturizer, urea, ammonium lactate or combinations thereof.

4. The method of claim 1, wherein the topical composition further comprises a penetration enhancer.

5. The method of claim 4, wherein the penetration enhancer is selected from dimethylsulfoxide (DMSO), methylsulfonylmethane (MSM), propylene glycol, dimethyl isosorbide, isopropyl myristate and combinations thereof.

6. The method of claim 1, wherein the composition is formulated in a dosage form selected from a cream, a lotion, a gel, an ointment, an emulsion, a solution, a suspension, an elixir, a tincture, a paste, a foam, an aerosol, a spray, a patch, a transdermal patch, a powder, a shake lotion, a solid, a sponge, a tape, a shampoo and applicator syringe (for intradermal injection).

7. (canceled)

8. The method of claim 1, wherein the cutaneous adverse effect is selected from maculo-papular rashes, inflammatory rashes, photosensitivity and secondary skin tumors.

9. The method of claim 1, wherein the cutaneous adverse effect is caused by cutaneous inflammation.

10. The method of claim 1, wherein the cutaneous adverse effect is caused by the upregulation of pro-inflammatory cytokines and/or chemokines.

11. The method of claim 1, wherein the method comprises once or twice daily topical application of therapeutically effective amounts of the said composition to the skin portion of the oncological therapy subject affected by the said cutaneous adverse effect until the cutaneous adverse effect is cured, prevented or alleviated or according to doctor's instructions.

12. (canceled)

13. (canceled)

14. (canceled)

15. The method of claim 6, wherein the method comprises once or twice daily topical application of therapeutically effective amounts of the said composition to the skin portion of the oncological therapy subject affected by the said cutaneous adverse effect until the cutaneous adverse effect is cured, prevented or alleviated or according to doctor's instructions.

16. The method of claim 2, wherein the topical composition further comprising an emollient, a moisturizer, urea, ammonium lactate or combinations thereof.

17. The method of claim 15, wherein the topical composition further comprises a penetration enhancer.

18. A method of treatment, prevention or alleviation of a cutaneous and/or mucosal adverse effect caused by oncological therapy selected from chemotherapy, immunotherapy, radiation therapy, targeted therapy, hormone therapy and stem cell transplant therapy, wherein the method comprises administering to an oncological therapy subject in need thereof a kit for topical administration comprising one or more dosage forms and instructions for use, wherein the dosage form comprises from about 0.01% to about 1% w/w, from about 1% to about 3% w/w, from about 1% to about 5% w/w, from about 3% to about 5% w/w tapinarof, or from about 5% to about 10% w/w tapinarof.
